(19) United States
(12) Patent Application Publication
(10) Pub. No.: US 2026/0306807 A1
(43) Pub. Date: Oct. 8, 2026

(54) Venticular Shunt System

(71) Applicant: Queen's University at Kingston, Kingston (CA)

(72) Inventors: Teresa Purzner, Kingston (CA); James Purzner, Kingston (CA)

(21) Appl. No.: 18/998,295

(22) PCT Filed: Jul. 25, 2023
(86) PCT No.: PCT/CA2023/050998

§ 371 Date: Jan. 24, 2025

Related U.S. Application Data
(60) Provisional application No. 63/392,184, filed on Jul. 26, 2022.

(51) Int. Cl. A61M 27/00; A61M 39/00; A61M 39/10; A61M 39/24

(52) U.S. Cl. CPC A61M 27/006; A61M 39/10; A61M 39/24; A61M 2039/0009; A61M 2205/04; A61M 2205/3331; A61M 2210/0693; A61M 2210/1017

(57) ABSTRACT

A ventricular shunt system allows for device replacement percutaneously, or, with one to two small incisions. The system includes a proximal catheter having one end held in a connector and the end of the proximal catheter capped by a screw cap which can be removed during a process of replacing the proximal catheter or for inserting devices to interrogate the interior of the proximal catheter. The connector allows communication between the interior of the proximal catheter through one side of a one-way valve to drain any excess cerebral spinal fluid that has built up in the ventricle system. One end of a distal catheter is connected to with the other side of the one-way valve and which is capped by a removable screw cap which can be removed during a process of replacing the proximal catheter or for inserting devices to interrogate the interior of the catheter.

### FIELD

The present disclosure relates to a ventriculoperitoneal shunt system which is configured to give clinician access to the interiors of the catheters forming part of the shunt system.

### BACKGROUND

Insertion and revision of ventriculoperitoneal shunts (VP shunts) are among the most commonly performed neurosurgical procedures in North America. VP shunts are cerebral shunts that are placed when there is an excessive buildup of cerebral spinal fluid (CSF), a disease state known as “hydrocephalus”. Hydrocephalus can result from a multitude of causes including infections, intraventricular hemorrhage, intracerebral hemorrhage, subarachnoid hemorrhage, strokes, brain tumors, leptomeningeal carcinomatosis, brain cysts, congenital malformations, aqueductal stenosis, arachnoid webs, neurodegenerative states like normal pressure hydrocephalus etc. The shunt itself is a thin tube that allows passage of CSF from the brain (ventricular spaces) to an alternative area in the body, thereby alleviating the fluid build-up. Flow is typically regulated by an in-line valve. Most commonly shunt systems are a ventriculoperitoneal, which drain to the abdominal cavity (>90%). Less common are ventriculo-thorax (0.6%), ventriculo-circulatory system (0.5%) and ventriculo-urinary (0.05%) systems (PMID: 15617596)

Unfortunately, VP shunts have high complication rates and require revision in approximately 15% of patients within the first 30 days and 20-40% of patients within the first year of insertion (PMID: 24089046). These rates are even higher in children, where longterm studies found only 15.5% of children with VP shunts did not require a revision (23101557). Shunt malfunctions can happen for a variety of reasons, most commonly catheter occlusion or infection, but also hardware malfunction/damage and many other sources. Despite advancements in shunt and shunt valve systems, rates of malfunction requiring revision remain much unchanged for the past 50 years (PMID: 18352802). Overall, ventricular shunts are estimated to be a billion-dollar-a-year cost to the US healthcare system (PMID: 15617596), equally split between shunt placements (43%) and shunt replacements (43%), the remainder being largely shunt removal (7%)

Currently, shunt revisions are invasive surgical procedures, commonly requiring a general anesthetic, placement of skull “pins” to fixate the head in space, intra-operative neuronavigation to ensure the correct trajectory of the cranial shunt catheter and an incision at both the cranial end of the shunt as well as the abdominal shunt catheter. For patients with repeat procedures or who have had previous abdominal operations, laparoscopic assistance for placement of the distal catheter is also considered. Patients with repeat operations can have multiple abdominal and cranial scars and with repeat procedures, risk of poor wound healing or catheter extrusion increases significantly. Similarly, risk of bowel perforation from the abdominal incision ranges from 0.5-3% (25837887) depending on the study, with increased risk relating to the number of times the abdomen is accessed. The operative time alone is on average 75 minutes (31874294 ), added to which is the anesthetic time (time to put the patient to put the patient under a general anesthetic and then to wake up and extubate following the procedure), time to register neuronavigation.

Patients with VP shunt malfunction present a diagnostic challenge in terms of identifying the source as well as site of malfunction. Furthermore, patients typically present with isolated headaches, and determining whether the headache is a reflection of a VPS malfunction, vs the many common causes of headaches, is often diagnostically challenging and can be impossible without direct interrogation of the shunt in the operating room. Collecting CSF from the shunt to investigate potential infection requires direct access to the shunt valve.

### SUMMARY

Disclosed herein is a ventriculoperitoneal shunts (VP) shunt device/system which allows for device replacement percutaneously, or, with 1-2 small incisions. The ventriculoperitoneal shunt device comprises a proximal catheter having a distal end configured to be inserted into the ventricle in the brain of a patient, and a proximal end section engaged by a first end of a proximal connector. A proximal end of the proximal end section is mated to a first removable screw cap. The shunt includes a second end of the proximal connector connected to a first side of a one-way valve. The proximal end section of the proximal catheter has an aperture located therein to provide a flow path from the proximal catheter into the proximal connector and into the one-way valve. A second side of the one-way valve is connected to a first end of a distal connector with a proximal end section of the distal catheter being engaged with the distal connector. A proximal end of the distal catheter is mated to a second removable screw cap. The proximal end section of the distal catheter has an aperture located therein to provide a flow path from the one-way valve into the distal connector and into the distal catheter. A distal end of the distal catheter configured to inserted into a body cavity in the patient spaced from the patient's brain so that any excessive cerebral spinal fluid drains from the patient's ventrical through the one-way valve down into the body cavity. The first and second screw caps being accessible and removable and replaceable by a clinician to access the interiors of the proximal and distal catheters.

The proximal connector may have a bent section, allowing variable angles and distances between the one-way valve and the first removable screw cap.

The proximal connector may extend substantially the length of the proximal catheter leaving an exposed distal end of said proximal catheter to provide increased flexibility and variability in length and angle of the exposed distal end of proximal catheter.

The proximal connector may be T-shaped such that the first screw cap is in-line, rather than perpendicular, with respect to the one-way valve.

The one-way valve, and proximal connector may be produced as an integral single unit.

The one-way valve, proximal connector and distal connector may be produced as an integral single unit.

The one-way valve and distal connector may be produced as an integral single unit.

The one-way valve, proximal connector and distal connector connectors may be produced as an integral single unit, and the first and second screw caps may be produced and embedded within the one-way valve.

The the first removable screw cap may be mated to a proximal end of the proximal catheter, and a proximal end of said distal catheter may be mated to the second removable screw cap.

A sheath may be used to envelop the distal catheter to allow for improved ease of distal catheter replacement.

This VP device may be utilized in conjunction with fluoroscopic guidance. The advantages of this approach is decreased operative time, may be performed in fluoroscopic suite rather than operating room, smaller incision size, decreased need for general anesthetic, decreased need for intra-operative cranial fixation, decreased need for intraoperative neuronaviagtion, decreased need for laparoscopic assistance, allows introduction of minimally invasive devices for removal or dissociation of material causing catheter blockade. The device may also assist in diagnosis of the source VP shunt malfunction through percutaneous injection of radio-opaque fluid to both the cranial and abdominal catheters, as well as through direct interrogation of both proximal and distal catheters. This device will be made with, and without, antibiotic-impregnation.

A further understanding of the functional and advantageous aspects of the present disclosure can be realized by reference to the following detailed description and drawings.

### BRIEF DESCRIPTION OF THE DRAWINGS

Embodiments will now be described, by way of example only, with reference to the drawings, in which:

FIG. 1 shows the current or prior art ventricular shunt system;

FIGS. 2a to 2i show various embodiments of a ventricular shunt system according to the present disclosure;

FIG. 3 shows different embodiments of a catheter valve communication used in the present ventricular shunt systems;

FIGS. 4a and 4b compares the prior art ventricular shunt system (FIG. 4a) with the present ventricular shunt system (FIG. 4b);

FIGS. 5a and 5b compares the procedure for installing the prior art ventricular shunt system (FIG. 5a) and the present ventricular shunt system (FIG. 5b);

FIG. 6 shows various embodiments of various catheter entrapment mechanisms for grasping, adjustment and/or removal of proximal and distal shunt catheters;

FIG. 7 shows various embodiments of catheter closure devices for the purpose of obstruction of CSF flow; protection and/or introduction of catheters, connectors and entrapment devices; securing proximal and distal catheters to a fixed position/connector, and/or prevention or catheter rotation;

FIG. 8 shows an embodiment of a catheter placement mechanism forming part of the present ventricular shunt system;

FIG. 9 shows various configurations that may be used for connector access for introduction, replacement or interrogation of the catheter, introduction of minimally invasive devices for removal or dissociation of material causing catheter blockade; and

FIG. 10 shows an access point radio-opaque marker.

FIG. 11a shows a representation of the present device with an obstruction in the proximal catheter but it may be in the distal catheter, or both.

FIG. 11b shows that to access the proximal catheter, the screw cap is removed at the proximal access port of the connector. For variations with percutaneous access, a screw cap is not required to be removed.

FIG. 11c illustrates that to identify a region of obstruction, a radio-opaque dye is directly injected into the catheter at the access point of the connector.

FIG. 11d shows that once an obstruction and it's location has been identified, the present minimally invasive device is then extended to the point of obstruction and the clot can be viewed, retrieval, or dissociated.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

Unless defined otherwise, all technical and scientific terms used herein are intended to have the same meaning as commonly understood to one of ordinary skill in the art.

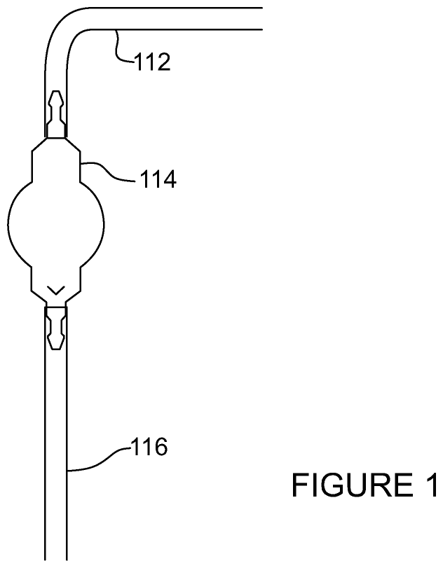

FIG. 1 shows the PRIOR ART ventricular shunt system currently used and includes a proximal catheter 112, which communicates between the patient's ventricular system and one side of valve 114, and distal catheter 116 that communicates between the other side of valve 114 and the patient's abdomen, thoracic cavity, or cardiovascular system, for example the heart or superior or inferior vena cava.

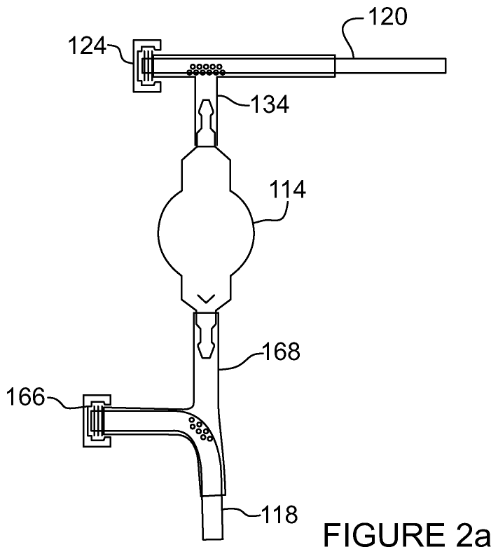

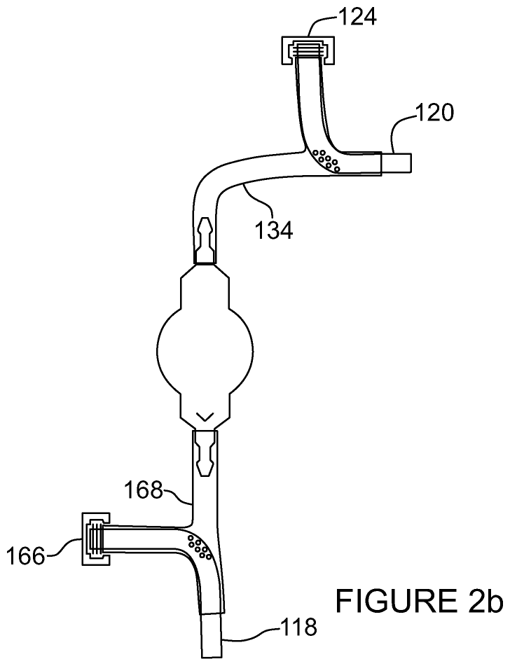

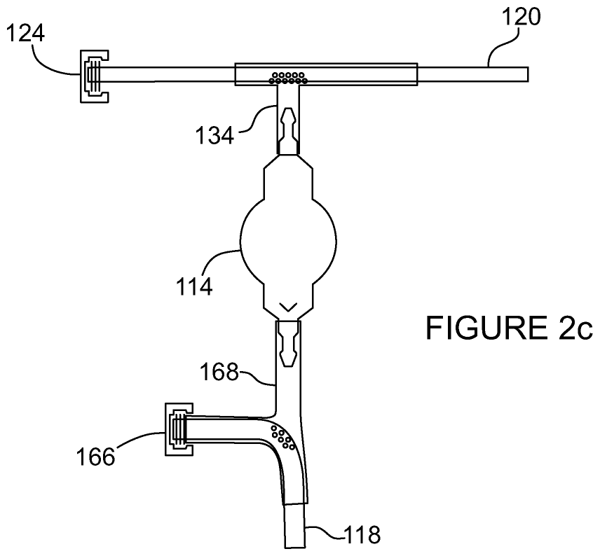

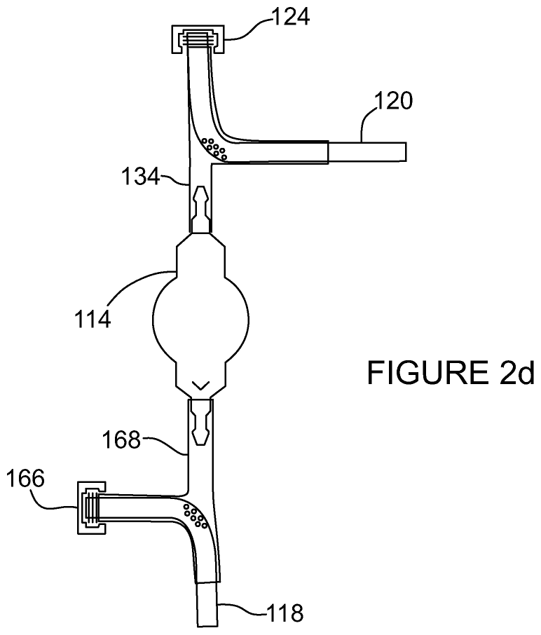

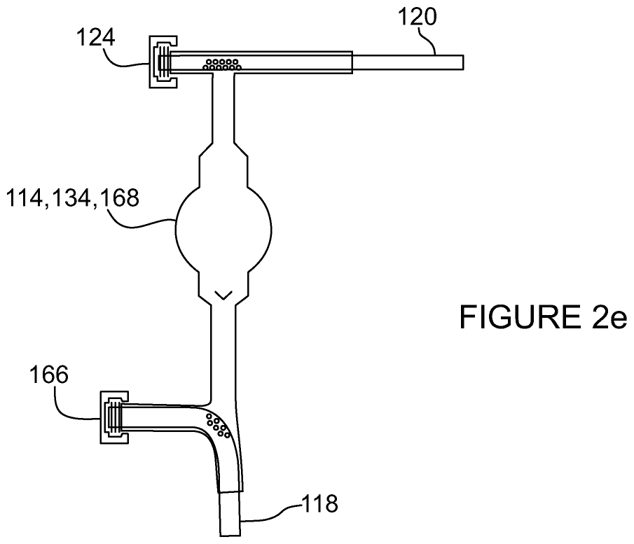

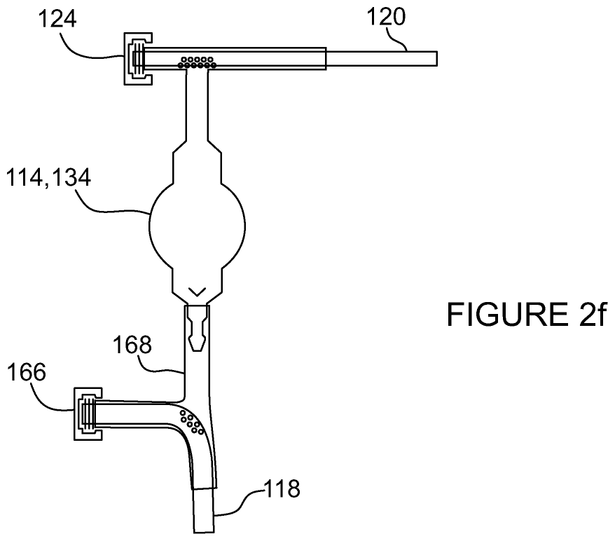

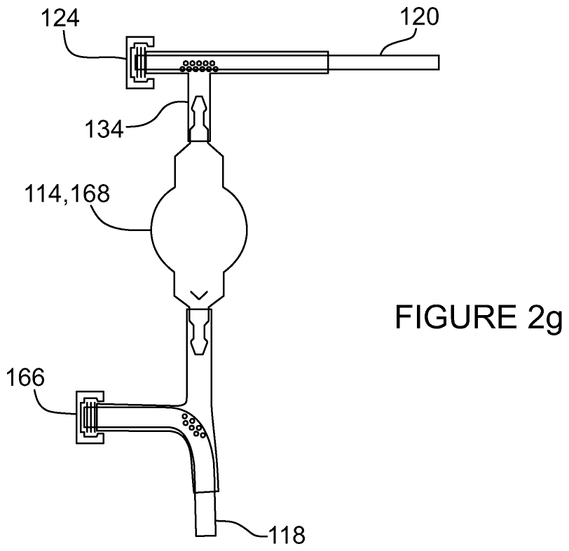

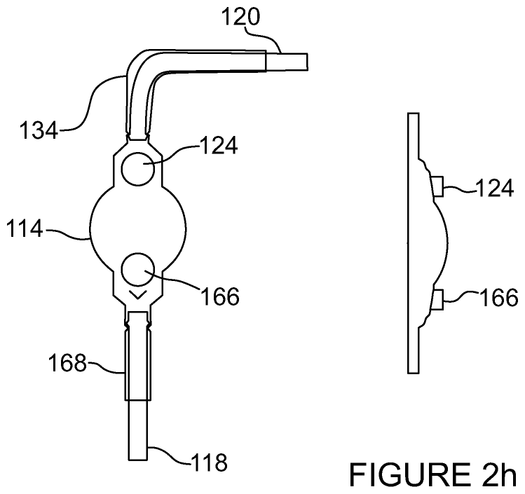

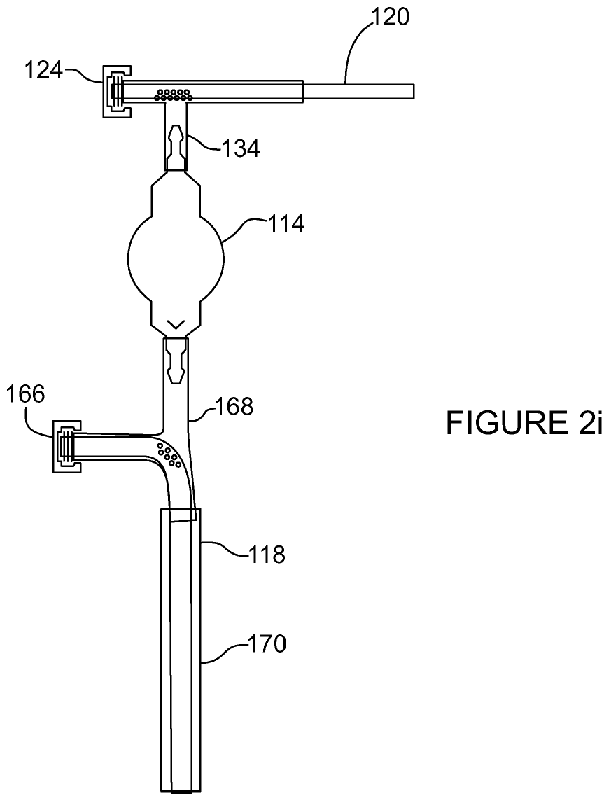

FIGS. 2a to 2j show various embodiments of the ventricular shunt system according to the present disclosure. FIG. 2a shows the basic shunt system comprised of the proximal catheter 120 having one end located in the patient's ventricle and the other end being capped by screw cap 124. The purpose screw cap 124 is to allow access to the interior of proximal catheter 120 in the event it needs to be replaced or otherwise interrogated, prevents egress of CSF and may serve as a means of engaging the catheter 120 and proximal connector 134, thereby fixing the components in place. The proximal catheter 120 is coupled to valve 114 by the proximal connector 134. A distal connector 168 is used to couple one end of a distal catheter 118 to the other side of valve 114. A second screw cap 166 is threaded onto the end of the distal catheter 118 and its purpose is to allow access to the interior of the distal catheter 118 in the event it needs to be replaced or otherwise interrogated, prevents egress of CSF and may serve as a means of engaging the catheter 120 and connector 134, thereby fixing the components in place. Catheters typically have an inner diameter of 1 to 1.5 mm and outer diameter of 1.3 to 3 mm. The entire shunt system, including screw caps and connectors, are buried under the skin and not directly visible.

A comparison of the currently used system of FIG. 1 and the new system of FIG. 2a disclosed herein reveals several significant advantages of the latter over the former.

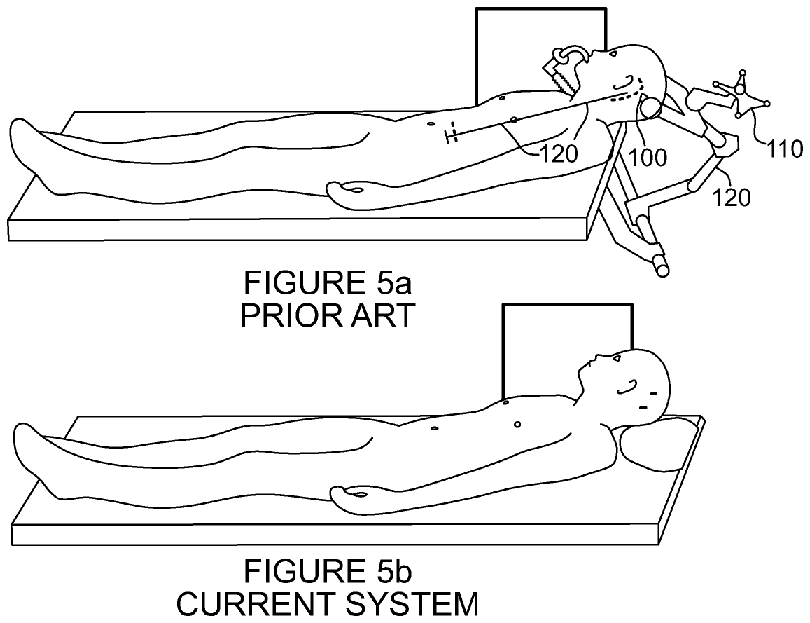

First, the prior system has the proximal catheter 112 and distal catheter 116 affixed to the valve 114. As a result, to replace either catheter, an open operation must be performed which requires a neurosurgeon and a full operating room suite with an anesthetist, scrub nurse, circulating nurse, other typical OR allied health members, typically a general anesthetic with endotracheal intubation, cranial fixation with a fixed headframe system and, commonly, intra-operative neuronavigation. The current design requires two incisions, typically 5 cm-10 cm in length. The first incision is made over the proximal catheter 112 and valve 114 through re-exposing the prior incision and dissection through the prior scar tissue, if possible. If skin over the prior incision is inadequately perfused due to extensive scar tissue or skin atrophy, then a new incision is created. The distal catheter 116 is replaced by creating a second, distal incision through the skin, subcutaneous tissue, muscle, peritoneum/pleura and into the abdominal or thoracic space. This incision is typically made remotely from the prior abdominal or thoracic incision, due to risk of intra-abdominal/thoracic scarring at the site of the previous incision which results in increased risk of injury to the bowel or lung. In the current system, a large-bore rod with a stylet, known as the “shunt passer” is then tunneled from the distal incision, subcutaneously through the abdominal and thoracic regions, through the anterior neck and finally past the attachment of the neck muscles to the skull, along the occipital bone of the skull and terminating at the cranial incision 1, shown in FIG. 5A.

In contrast, in the system disclosed herein, the proximal catheter 120 and distal catheter 118 can be directly accessed through the proximal connector 134 and distal connector 168 percutaneous or through one or two small stab incisions (approximately 1 cm) through the scalp over the proximal screw cap 124 and distal screw cap 166. The minimally invasive access may allow catheters to be replaced in an interventional radiology suite by either a neurosurgeon or interventional neuroradiologist without requirement of a general anesthetic and endotracheal intubation, cranial fixation, neuronavigation, nor creation of new abdominal incisions or tunneling of a shunt passer.

Secondly, the current system, access to the proximal and distal catheter through the open surgical approach, as described above, allows for direct visualization of only several centimeters of the proximal catheter 120 and distal catheter 118 closest to the valve. In the current system, the minimally invasive approach to accessing the proximal and distal screw caps allows for fluoroscopic interrogation of the entire length of the catheters (up to about 120 cm) and thereby assessment of the specific region of blockage (for example, through injection of radio-opaque dye). The minimally invasive approach also allows for introduction of an array of minimally invasive tools which may allow for direct visualization, removal or dissociation of the source of blockage, thereby potentially avoiding the necessity of exchanging the catheter.

FIGS. 2b to 2j show variations on the structure of FIG. 2a. For example, FIG. 2b differs from FIG. 2a in that the proximal connector 134 has a bend, allowing variable angles and distance between the valve 114 and the access point 124.

FIG. 2c differs from FIG. 2a in that the proximal connector 134 does extend the length of the proximal catheter 120. The exposed proximal catheter 120 can therefore have increased flexibility and variability in length and angle.

FIG. 2d differs from FIG. 2a in that the proximal connector 134 is T-shaped such that the access point 124 is in-line, rather than perpendicular, to the valve 114. The in-line vs perpendicular alignment has differing advantages and disadvantages in terms of guidewire access and CSF flow dynamics.

FIG. 2e differs from FIG. 2a in the valve 114, proximal 134 and distal 168 connectors are a single unit. This allows for increased stability of the construct as a whole, but decreased flexibility in patient-to-patient variability.

FIG. 2f differs from FIG. 2a in that the valve 114 and proximal connector 134 are a single unit. This allows for increased stability of the construct as a whole, but decreased flexibility in patient-to-patient variability.

FIG. 2g differs from FIG. 2a in that the valve 114 and distal connector 168 are a single unit. This allows for increased stability of the construct as a whole, but decreased flexibility in patient-to-patient variability.

FIG. 2h differs from FIG. 2a in that the valve 114, proximal 134 and distal connectors 168 are a single unit. The proximal and distal access points 124 and 166 are embedded within the valve 114 rather than a second arm in the proximal 134 and distal 168 connectors. This allows for increased stability of the construct as a whole and, potentially, improved CSF flow dynamics, but decreased flexibility in patient-to-patient variability.

FIG. 2i differs from FIG. 2a in that a sheath 170 is placed over the distal catheter 168 to allow for improved ease of distal catheter replacement.

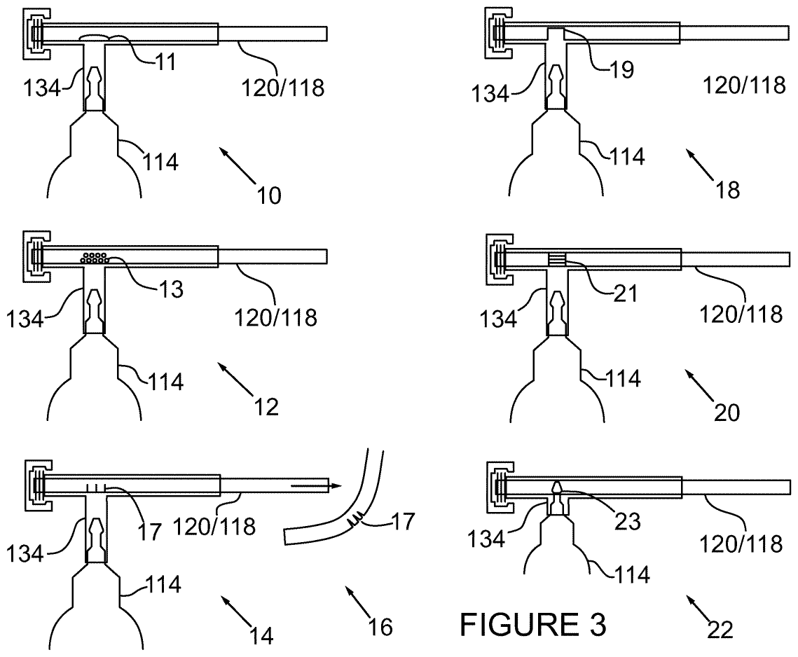

FIG. 3 shows different embodiments of a catheter valve communication used in the present ventricular shunt systems with the embodiment 10 showing the end section of the proximal catheter 120 or distal catheter 118 that couples to the valve 114. The defect 11 (or space) is the space through which the cerebral spinal fluid (CSF) flows from the catheter 120 to the valve and/or connector, through the proximal connector 134.

Embodiment 12 shows an embodiment wherein fenestrations/perforations 13 are shown at the valve-facing end of the proximal and distal catheter 118. These fenestrations/perforations are to allow CSF to flow between the catheter 120, connector 134 and/or valve 114.

Embodiment 14 and 16 shows an embodiment of a distal catheter wherein linear defects 17 are shown at the valve-facing end of the proximal and distal catheter 14. A. These linear defects 17, when bent, create gaps which allow CSF to flow between the catheter 120 to the valve 114 via the connector 134.

Embodiment 18 shows an embodiment wherein a linker 19 is shown at the valve-facing end of the proximal and distal catheter. This linker 19 provides a defect which allow CSF to flow between the catheter 120 to the valve 114 via the connector 134.

Embodiment 20 shows an embodiment wherein wires 21 are shown at the valve-facing end of the proximal and distal catheter. These wires permit a space which allow CSF to flow between the catheter 120 to the valve to the valve 114 via the connector 134.

Embodiment 22 shows a perforation 23 at the valve-facing end of the proximal and distal catheter. This perforation permits the valve to be inserted directly into the proximal and distal catheter, allowing CSF to flow between the catheter 120 to the valve to the valve 114 via the connector 134.

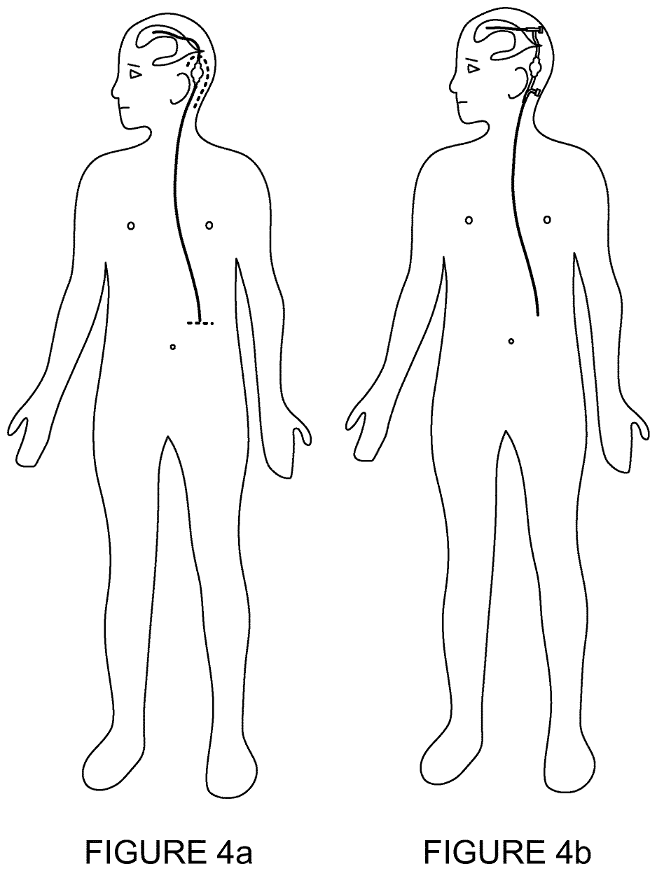

FIG. 4A shows prior art in situ as well as incisions 1 and 2 required for revision of the current or prior art shunt system. There are two incisions. The first incision 1 is typically a frontal or parieto-occipital incision sufficiently large for placement of a burr hole for insertion of the proximal catheter, placement of the valve and connection of the valve to both the proximal and distal catheter. The second incision 2 is made in the abdomen or thorax, to allow for insertion of the distal catheter into the abdominal or thoracic cavity, as well as introduction of the subcutaneous shunt passer, which is a large-bore rod which allows subcutaneous passage of the distal catheter from the valve to the abdominal or thoracic cavity. The prior art shunt components include a proximal catheter, distal catheter and valve as shown in FIG. 1.

FIG. 4B demonstrates the present system in situ as well as stabs required for revision of the ventricular shunt system disclosed herein involves. Incisions 1 and 2, cranial and abdominal respectively, in FIG. 4A are replaced by small cranial stabs 1 and 2. The first cranial stab 1 is made over the proximal connector, allowing for grasping, readjustment, interrogation, introduction of minimally invasive tools and removal and/or replacement of proximal catheter. The second stab 2 is also cranial and is made over the distal connector, allowing for grasping, readjustment, interrogation, introduction of minimally invasive tools and removal and/or replacement of distal catheter.

There is no abdominal incision. Shunt components include proximal catheter, proximal connector, valve, distal connector and distal catheter.

Referring to the bottom part of FIG. 5a, the process of replacement of the prior art ventricular shunt system is illustrated. Replacement typically requires a general anesthetic with endotracheal intubation, cranial fixation with a fixed headframe system, intra-operative neuronavigation and a full operating room suite with an anesthetist, scrub nurse, circulating nurse, neurosurgeon and other typical OR allied health members. Once sedated and intubated, the patient is placed supine on the operating room table with a large shoulder roll and head rotated away from the side of the shunt. Cranial pins 100 (only one is shown in FIG. 5A) are used to fix the skull in place which form part of a cranial head fixation system 102. Pre-operative imaging is uploaded into a neuronavigation device 110 and registered to the patient. A cranial incision 1 is made over the proximal catheter, valve and distal catheter, if possible, through re-exposing the prior incision and dissection through the prior scar tissue. If skin over the prior incision is inadequately perfused due to extensive scar tissue or skin atrophy, then a new incision is created.

The proximal catheter is disconnected from the vale and spontaneous CSF flow is determined. The distal tubing is similarly disconnected from the valve system. Distal flow can be assessed by several methods, including subjective assessment of resistance to an injection of saline. The proximal and/or distal catheter is then removed. The proximal catheter is replaced by re-passing a new proximal catheter under guidance of the neuronvaigation system 110. The distal catheter is replaced by creating a second, distal incision 2 through the skin, subcutaneous tissue, muscle, peritoneum/pleura and into the abdominal or thoracic space. This incision 2 is typically made remotely from the prior incision, due to risk of intra-abdominal/thoracic scarring at the site of the previous incision which results in increased risk of injury to the bowel or lung. A large-bore rod 120 with a stylet, known as the “shunt passer” is then tunneled from the distal incision, subcutaneously through the abdominal and thoracic regions, through the anterior neck and finally past the attachment of the neck muscles to the skull, along the occipital bone of the skull and terminating at the cranial incision 1, shown in FIG. 5A. The stylet is removed and the distal tubing is passed through the center bore of the rod.

The rod is removed and the distal catheter is then placed into the abdominal or pleural cavity under direct vision. Incisions 1 and 2 are sutured closed in layers.

Referring to FIG. 5B, the process of replacing the ventricular shunt system using the present device is disclosed herein. The present system is designed so that under most circumstances, replacement of the ventricular shunt system does not require general anesthetic, does not require endotracheal intubation and does not require cranial fixation, intra-operative neuronavigation or a full operation suite, does not require re-opening of previous proximal or distal incisions and does not require a shunt passer. However, clinicians skilled in the art will appreciate that there may be circumstances where some or all of these may be needed. Under conscious sedation, the patient is placed supine in a fluoroscopy suite. A stab incision 1 is made over the proximal connector and the connector cap is removed. The proximal shunt is interrogated through injection of radio-opaque dye. The distal shunt is similarly accessed through a second stab incision 2 over the distal connector and interrogated with a radio-opaque dye. The blockage is identified and a guide wire is placed along the blocked catheter under direct fluoroscopic visualization. The catheter is then removed over the wire and a new catheter is similarly passed over the guide wire. The small stab incision is then closed with sutures, if required.

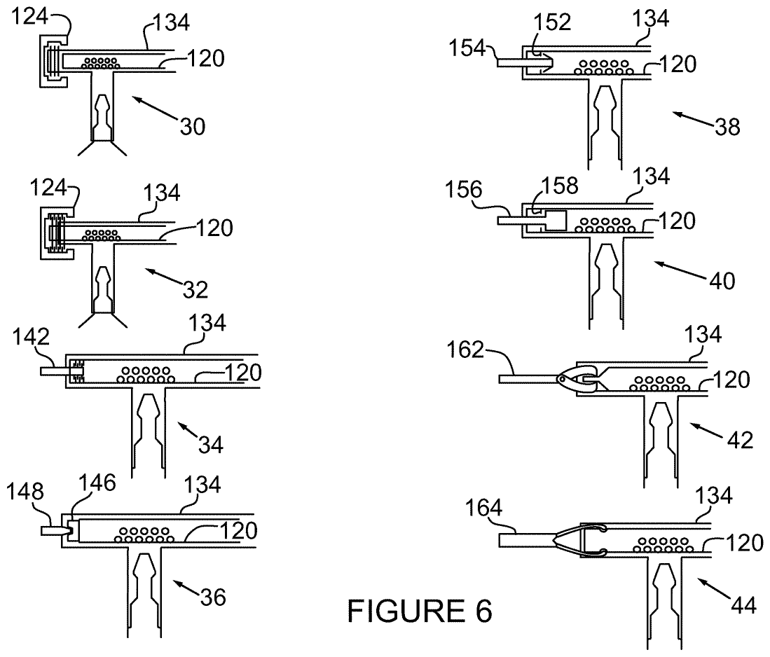

FIG. 6 shows various embodiments of various catheter entrapment mechanisms in embodiments 30, 32, 34, 36, 38, 40, 42 and 44. Embodiment 30 shows threaded cap 124 which engages with the threads of the connector 134. Screwing on the cap prevents CSF to egress from the connector.

Embodiment 32 shows threaded cap 124 which engages with the threads of both the valve facing end of the catheter 120 and the connector 134. Screwing on the cap 124 engages both catheter 120 and connector 134, thereby fixing the three components in place and preventing egress of CSF. When cap 124 is removed, the catheter 120 can be directly grasped and removed under direct visualization.

Embodiment 34 shows the valve facing end of the catheter 120 is internally threaded. A threaded screwdriver 142 is then used to engage with the internal threading of the catheter 120 and thereby entrap and remove or readjust the catheter 120.

Embodiment 36 shows a screwhead 146 at the termination of the valve facing end of the catheter 120. The screwhead 146 (for example, slotted, phillips, quadrex, square, torx, hex, tri-wing, spanner or variations thereof) is engaged with a screwdriver 148 appropriate for the screwhead. The screwdriver 148 is used to engage with screw head 146 and thereby release and remove or readjust the catheter 120.

Embodiment 38 shows an internal protrusion near the termination of the valve facing end of the catheter 120. A deployable device 154 is then used to engage with the protrusion 152 and thereby entrap and remove or readjust the catheter 120.

Embodiment 40 shows a protrusion 158 near the termination of the valve facing end of the catheter 120. A device with a deployable balloon 156 is then passed through the termination of the valve facing end of the catheter 120 and inflated. Once inflated, the balloon 156 engages with the protrusion 158 and thereby entrap and remove or readjust the catheter 120.

Embodiments 42 and 44 show articulated graspers 162 and 164 respectively. The graspers 162 and 164 are used to directly grasp (embodiment 42) or engage (embodiment 44) with the termination of the valve facing end of the catheter 120 and thereby entrap and remove or readjust the catheter 120.

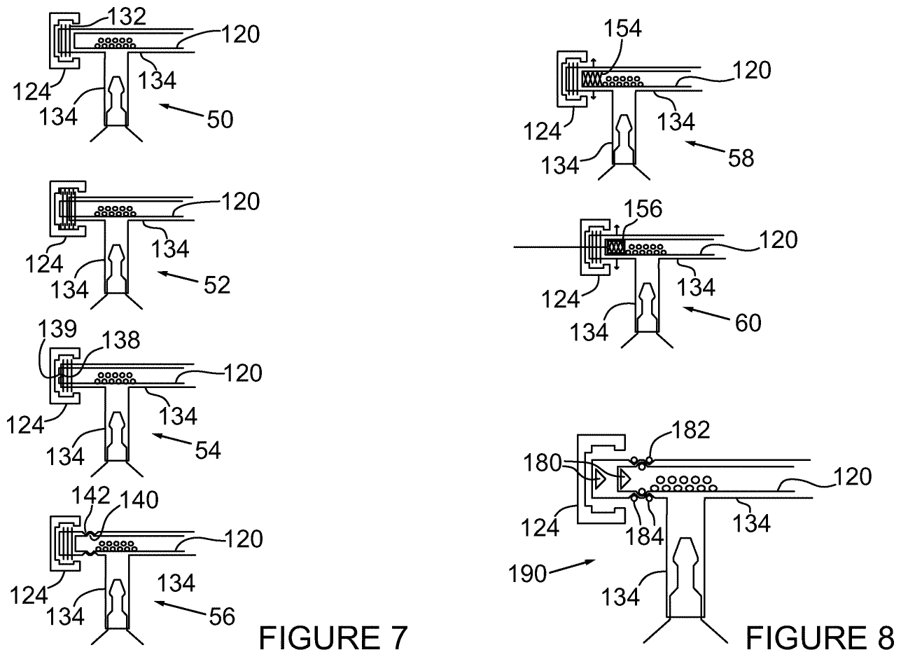

FIG. 7 shows various embodiments 50, 52, 54, 56, 58 and 60 of catheter closure devices. Shown generally at 50 is threaded cap 124 configured to engage with the threads 132 of the connector 134. Screwing on the cap 124 prevents CSF to egress from the T-connector 134 contained the end section of proximal catheter 120.

Shown generally at 52 is an embodiment of the end of the catheter 120 that is threaded with threaded cap 124 shown engaging with the threads of both the threaded valve facing end of the catheter 120 and the connector 134. Screwing on the cap 124 engages both catheter 120 and connector 134, thereby fixing the three components in place and closing the system to prevent egress of CSF.

Shown generally at 54 is threaded cap 124 configured to engage with the threads of the connector 134. A wedge-shaped protrusion 138 in the end of connector 134 interlocks with a corresponding wedge-shaped impression 139 in the end of catheter 120, thereby fixing the valve facing end of the catheter 120 and the connector 134 in place. The wedge lock and cap 124 together both fix the cap 124, connector 134 and catheter 120 in place and close the system to prevent egress of CSF.

Shown generally at 56 is threaded cap 124 configured to engage with the threads of the connector 134. A ring-like or mound-like protrusion 140 at the end of catheter 120 interlocks with a correlative indentation 142 in the wall of connector 134, thereby fixing the valve facing end of the catheter 120 and the connector 134 in place. The interlocking protrusions and cap 124 together both fix the cap 124, connector 134 and catheter 120 in place and close the system to prevent egress of CSF.

Shown generally at 58 is threaded cap 124 configured to engage the threads of the connector 134. A self-deploying mechanism 154 results in expansion of the valve facing end of the catheter 120, thereby fixing it in place within the fixed-diameter connector 134. The self-deploying mechanism and cap together both fix the cap 124, connector 134 and catheter 120 in place and close the system to prevent egress of CSF.

Shown generally at 60 is threaded cap 124 which engages with the threads of the connector 134. A balloon-assisted device 156 results in expansion of the valve facing end of the catheter 120 upon inflation of the balloon, thereby fixing it in place within the fixed-diameter connector 134. The balloon-assisted mechanism 156 and cap 124 together both fix the cap 124, connector 134 and catheter 120 in place and close the system.

FIG. 8 shows an embodiment of a catheter placement mechanism 190 forming part of the present ventricular shunt system, in which radio-opaque markers are in the connector and/or catheter 120. Radio-opaque markers may be used for rotational alignment (arrows 180) and engagement of the valve facing end of the catheter (dots 182) within the connector (dots 184). Some variance of the radio-opaque markers may be used in both the proximal and distal connectors and catheter systems to guide access to the ventricular shunt system for shunt interrogation, for ensuring appropriate engagement and closure of the catheter within the connector, and/or to access the ventricular shunt system for catheter removal or replacement. The markers may be used for any of the closure devices in FIG. 7 and access devices in FIG. 9.

FIG. 9 shows different views 74 to 90 of devices for connector access for introduction, replacement or interrogation. A stab incision in the skin 150 of the patient over the screw cap 124 is shown generally at 74 which allows direct access to the screw cap 124, catheter 120 and connector components 128 located under the patient's skin. The screw cap 124 can then be directly removed to access the connector 134 and proximal catheter 120.

Embodiments 76 to 82 demonstrate various embodiments of a percutaneous approach in which a stylet or needle 130 is introduced over the screw cap 124. A series of progressively larger dilators are threaded over the stylet 130 (a single dilator 140 is shown generally at 78 while dilator 140 is located inside a larger dilator 142 as shown at 80) until a sufficient diameter is achieved needed to access the screw cap 124, connector 134 catheter 120, as shown generally at 82. The screw cap 124 can then be removed to access the connector 134 and catheter 120.

The views from 84 to 90 demonstrates a percutaneous approach in which stylet or needle 130 is introduced through the skin into an access point within the screw cap or connector as shown generally at 84. A sheath 160 is threaded over the stylet or needle 130 to access the connector as shown generally at 86. Once the sheath 160 is in place, the stylet 130 is removed as shown at 88. The sheath 160 can be used directly to interrogate the catheter 120, or, a guidewire 170 and/or a series of diagnostic catheters can be used to access the ventricular shunt system as shown generally at 90.

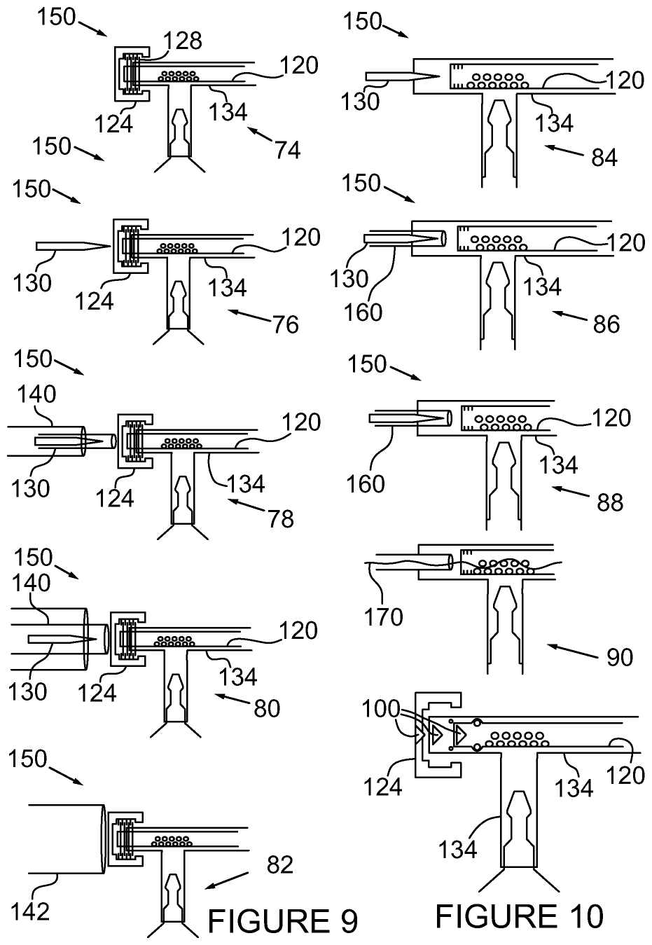

FIG. 10 shows an access point radio-opaque marker 100 in the screw cap 124, connector and/or catheter. Radio-opaque markers can identify the specific location of the cap 124, connector 134, catheter 120 and their components under fluoroscopic guidance. The radio-opaque markers can thereby be used to guide open or percutaneous access to the shunt access point 124. Radio-opaque markers can also be used for direction of needles, stylets, dilators, catheters, guidewires and other radio-opaque instruments and dyes necessary for placement, adjustment and interrogation of the catheter system.

Since whole procedure is done under Xray guidance (fluoroscopy), there is needed a device that is radio-opaque to provide guidance to know where to stab, and how to introduce wires, adjust catheters etc. Since these components cannot be seen directly by the clinician since they are all under the skin, markers that can be observed by X-rays are needed so the clinician can see what they are doing.

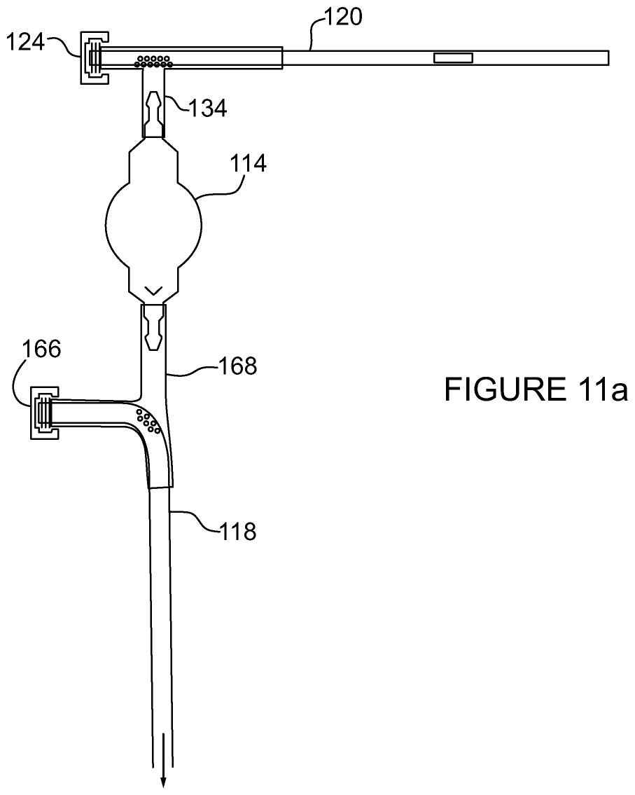

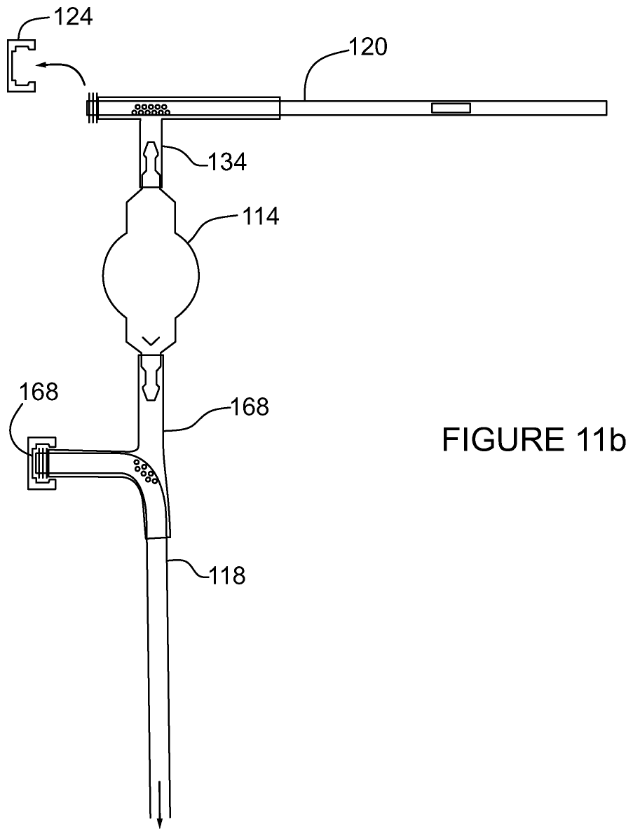

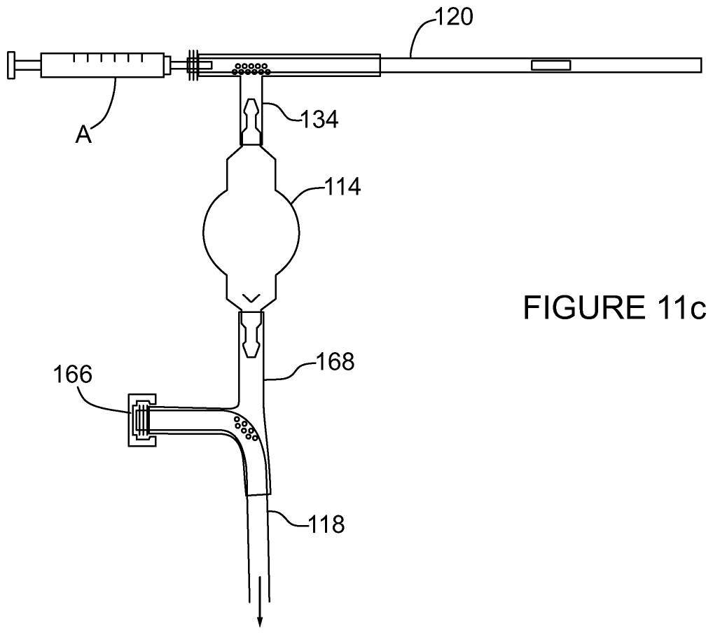

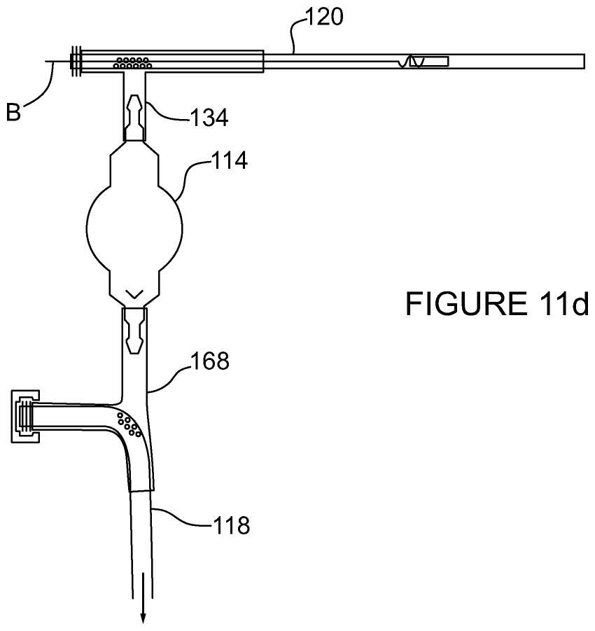

FIGS. 11a to 11d shows an example of the minimally invasive approach to clot viewing, retrieval and dissociation and shows the steps through which clot viewing, retrieval or dissociation can be performed. First the catheter is accessed by either removal of the cap or percuateneous approaches as described in FIG. 9. Next, a radio-opaque dye A is injected to localize the source of and obstruction H. Finally, the clot viewing, retrieval or dissociation device B is inserted directly through the catheter and the clot is retrieved. The catheter system is then flushed out and reassessed for patency, as in FIG. 11c.

Therefore what is claimed is:

1. A ventriculoperitoneal shunt device, comprising:
a proximal catheter (120) having a distal end configured to be inserted into the ventricle in a patient's brain, and a proximal end section of said proximal catheter (120) engaged by a first end of a proximal connector (134), a first removable screw cap (124) in communication with said proximal catheter (120);
a second end of the proximal connector (134) connected to a first side of a one-way valve (114), the proximal end section of the proximal catheter (120) having an aperture located therein to provide a flow path from the proximal catheter (120) into the proximal connector (134) and into the one-way valve (114);
a second side of the one-way valve (114) being connected to a first end of a distal connector (168), a proximal end section of the distal catheter (118) being engaged with the distal connector (118), a proximal end of said distal catheter (118) being mated to
a second removable screw cap (166) in communication with said, the proximal end section of the distal catheter (118) having an aperture located therein to provide a flow path from the one-way valve (114 into the distal connector (168) and into the distal catheter (118), a distal end of said distal catheter (118) configured to be inserted into a body cavity in said patient spaced from the patient's brain so that any excessive cerebral spinal fluid drains from the patient's ventrical through the one-way valve (114) down into the body cavity; and
said first and second screw caps (124, 166) being accessible and removable and replaceable by a clinician to access interiors of the proximal and distal catheters (120, 118).

2. The ventriculoperitoneal shunt device according to claim 1, wherein the proximal connector (134) has a bent section, allowing variable angles and distances between the one-way valve (114) and the first removable screw cap (124).

3. The ventriculoperitoneal shunt device according to claim 1, wherein the proximal connector (134) extends substantially the length of the proximal catheter (120) leaving an exposed distal end of said proximal catheter (120) to provide increased flexibility and variability in length and angle of the exposed distal end of proximal catheter (120).

4. The ventriculoperitoneal shunt device according to claim 1, wherein the proximal connector (134) is T-shaped such that the first screw cap (124) is in-line, rather than perpendicular, with respect to the one-way valve (114).

5. The ventriculoperitoneal shunt device according to claim 1, wherein the one-way valve (114), and proximal connector (134) are an integral single unit.

6. The ventriculoperitoneal shunt device according to claim 1, wherein the one-way valve (114), proximal connector and distal connector (168) connectors are an integral single unit.

7. The ventriculoperitoneal shunt device according to claim 1, wherein the one-way valve (114) and distal connector (168) connectors are an integral single unit.

8. The ventriculoperitoneal shunt device according to claim 1, wherein the one-way valve (114), proximal connector (134) and distal connector (168) connectors are an integral single unit, and wherein the first and second screw caps (124, 166) are embedded within the valve (114).

9. The ventriculoperitoneal shunt device according to claim 1, wherein the first removable screw cap (124) is mated to a proximal end of said of said proximal catheter (120), and wherein a proximal end of said distal catheter (118) is mated to said second removable screw cap (166).

10. The ventriculoperitoneal shunt device according to claim 1d, wherein a sheath (170) envelops the distal catheter (168) to allow for improved ease of distal catheter replacement (168).
